(19) United States
(12) Patent Application Publication
(10) Pub. No.: US 2009/0171465 A1
(43) Pub. Date: Jul. 2, 2009

(54) Polymeric Regions For Implantable Or Insertable Medical Devices

(75) Inventors: Weenna Bucay-Couto, Burlington, MA (US); Jianmin Li, Lexington, MA (US)

Correspondence Address: MAYER & WILLIAMS PC, 251 NORTH AVENUE WEST, 2ND FLOOR, WESTFIELD, NJ, 07090, US

(73) Assignee: Boston Scientific Scimed, Inc., Maple Grove, MN (US)

(21) Appl. No.: 12/276,938

(22) Filed: Nov. 24, 2008

Related U.S. Application Data
(60) Provisional application No. 61/009,377, filed on Dec. 28, 2007.

(51) Int. Cl. A61F 2/82

(52) U.S. Cl. 623/23.7; 623/23.71

(57) ABSTRACT

According to one aspect, the present invention is directed to implantable or insertable medical devices which comprise polymeric regions that comprise high vinyl acetate content EVA (or another soft polymer) and a therapeutic agent. In another aspect, the present invention is directed to implantable or insertable medical devices which comprise (a) a first region comprising a first EVA and (b) a second region adjacent to the first region that is of lower durometer than the first region.

### STATEMENT OF RELATED APPLICATIONS

This application claims the benefit of U.S. Provisional Patent Application Ser. No. 61/009,377, filed Dec. 28, 2007, entitled “Polymeric Regions For Implantable Or Insertable Medical Devices”, which is incorporated in its entirety by reference herein.

### TECHNICAL FIELD

This invention relates to medical devices, more particularly to implantable or insertable medical devices having polymeric regions.

### BACKGROUND INFORMATION

Numerous polymer-based medical devices have been developed for implantation or insertion into the body. For example, in recent years, drug eluting coronary stents, which are commercially available from Boston Scientific Corp. (TAXUS), Johnson & Johnson (CYPHER) and others, have become the standard of care for maintaining vessel patency. These existing products are based on metallic balloon expandable stents with biostable polymer coatings, which release antiproliferative drugs at a controlled rate and total dose.

As another example, polymeric ureteral stents are widely used to facilitate drainage in the upper urinary tract (e.g., drainage from the kidney to the bladder). They are used, for example, in post endo-urological procedures to act as a scaffold in the event of ureteral obstruction secondary to the procedure. Ureteral stents are also used as palliative devices to provide patency in the presence of congenital defects, strictures or malignancies, as well as in other instances where ureteral obstruction may occur. A schematic illustration of a ureteral stent 10 in accordance with the prior is illustrated in FIGS. 1A and 1B. The stent 10 has a proximal (bladder) end 10p and a distal (renal) end 10d. It is a tubular polymer extrusion having a shaft 12, a distal renal retention structure (e.g., renal “pigtail” 14), and a proximal bladder retention structure (e.g., bladder “pigtail” 16). These retention structures prevent upward migration of the stent toward the kidney or downward migration of the stent toward the bladder. The shaft 12 in cross-section is a single extruded layer as seen from FIG. 1A, which is taken along line b-b of FIG. 1A. Once properly deployed in the ureter, the stent 10 provides ureteral rigidity and allows the passage of urine. The stent 10 of FIGS. 1A and 1B is further provided with the following features: (a) a tapered tip 11, to aid insertion, (b) multiple side ports 18 (one numbered), which are arranged in a spiral pattern down the length of the body to promote drainage, (c) graduation marks 25 (one illustrated) for visualization by the physician to know when the appropriate length of stent has been inserted into the ureter, and (d) a suture 22, which aids in positioning and withdrawal of the stent. During placement, such ureteral stents 10 are typically placed over a urology guide wire, through a cystoscope and advanced into position with a positioner. Once the distal (renal) end of the stent is advanced into the kidney/renal calyx, the guide wire is removed, allowing the pigtails 14, 16 to form in the kidney 19 and bladder 20, as shown in FIG. 2. The renal pigtail 14 of the stent may be closed or tapered, depending on the method of insertion (e.g., the use of a guide wire or otherwise). As shown in FIG. 2, the stent 10 extends through the ureteral orifice 21a and into the bladder 20. For clarity, the ureter entering bladder 20 through the opposite ureteral orifice 21b is not shown.

Ureteral stents are known to be associated with a degree of pain and/or discomfort, particularly in the bladder and flank area after insertion. One way of addressing this pain is to use a softer material, particularly in forming the proximal (bladder) end of the stent, which engages more sensitive tissue. Extrusion can be done using a firm durometer EVA (e.g., Elvax® 460) at the distal end (i.e., kidney end), which improves stent placement, and a soft durometer EVA (e.g., Elvax® 250) at the proximal end (i.e., bladder end), which improves comfort. A specific example of such a stent is the Polaris™ Dual Durometer Percuflex® Ureteral Stent with HydroPlus™ Coating, available from Boston Scientific, Natick, Mass., USA. Other ways of addressing pain and discomfort include providing systemically administered painkillers or providing devices which release painkillers locally. See, e.g., Pub. No. US 2006/0264912 A1 entitled “Medical devices for treating urological and uterine conditions.”

Another issue associated with ureteral stents is the formation of encrustation in vivo, which may be addressed, for example, through the use of devices that release antimicrobial compounds locally. In this regard, see, e.g., Pub. No. 2004/0249441 A1 entitled “Implantable or insertable medical device resistant to microbial growth and biofilm formation.”

### SUMMARY OF THE INVENTION

According to one aspect, the present invention is directed to implantable or insertable medical devices which comprise polymeric regions that comprise high vinyl acetate content EVA (or another soft polymer) and a therapeutic agent.

In another aspect, the present invention is directed to implantable or insertable medical devices which comprise (a) a first region comprising EVA and (b) a second region adjacent to the first region that is of lower durometer than the first region. For example, the implantable or insertable medical devices may comprise (a) a first region comprising a first EVA and (b) a second region adjacent to the first region that comprises a second EVA that has a vinyl acetate content that is higher than that of the first EVA.

These and other aspects, as well as various embodiments and advantages of the present invention will become readily apparent to those of ordinary skill in the art upon review of the Detailed Description to follow.

### BRIEF DESCRIPTION OF THE DRAWINGS

FIG. 1A is a schematic illustration of a ureteral stent, according to the prior art. FIG. 1B a schematic cross-sectional view, taken along line b-b of FIG. 1A.

FIG. 2 shows the ureteral stent of FIGS. 1A-1B as positioned within the body.

FIG. 3A is a schematic illustration of a ureteral stent, according to an embodiment of the present invention. FIG. 3B a schematic cross-sectional view, taken along line b-b of FIG. 3A.

FIG. 4A is a schematic illustration of a vascular stent in accordance with an embodiment of the present invention. FIG. 4B is a schematic cross-sectional view, taken along line b-b of FIG. 4A.

FIG. 5 is a plot of cumulative release of tamsulosin as a function of time for various EVA samples imbibed with tamsulosin.

### DETAILED DESCRIPTION OF THE INVENTION

A more complete understanding of the present invention is available by reference to the following detailed description of numerous aspects and embodiments of the invention. The detailed description of the invention which follows is intended to illustrate but not limit the invention.

In one aspect, the present invention is directed to implantable or insertable medical devices which comprise polymeric regions (e.g., layers, monolithic regions, etc.) that comprise high vinyl acetate content EVA (or another soft polymer) and a therapeutic agent.

“Therapeutic agents”, “pharmaceuticals,” “pharmaceutically active agents”, “drugs” and other related terms may be used interchangeably herein and include genetic therapeutic agents, non-genetic therapeutic agents and cells. Therapeutic agents may be used singly or in combination.

As used herein, a “polymeric” region is one that contains polymers, for example, 50 wt % or less to 75 wt % to 90 wt % to 95 wt % to 97.5 wt % to 99 wt % polymers, or more.

As used herein a “low durometer polymeric region” has a durometer ranging from 65 to 70 to 75 to 77 to 80 to 83 to 85 (Shore A). The relative amounts of ethylene and vinyl acetate in EVA affect its durometer, and the EVA employed in low durometer polymeric regions are typically high vinyl acetate content EVA. As used herein “high vinyl acetate content” EVA typically has vinyl acetate contents ranging from 25 to 30 to 35 to 40 wt %. One example of high vinyl acetate content EVA is Elvax® 250 with a vinyl acetate content of about 28 wt % and a durometer of about 80 (Shore A).

Conversely, as used herein an “elevated durometer region” has a durometer ranging from 80 to 85 to 90 to 95 to 100 (Shore A), more preferably 85 to 87 to 90 to 93 to 96 Shore A. The EVA employed in elevated durometer polymeric regions are typically low vinyl acetate content EVA. As used herein “low vinyl acetate content” EVA typically has a vinyl acetate content ranging from 1 to 5 to 10 to 15 to 20 to 25 to 30 to 35 wt %, more preferably 7.5 to 18 wt %. One example of low vinyl acetate content EVA is Elvax® 460 with a vinyl acetate content of about 18 wt % and a durometer of about 90 (Shore A).

In another aspect, the present invention is directed to implantable or insertable medical devices which comprise (a) a first region comprising EVA and (b) a second region adjacent to the first region that is of lower durometer (softer) than the first region.

For example, the implantable or insertable medical devices may comprise (a) a first region comprising a first EVA and (b) a second region adjacent to the first region that comprises a second EVA that has a vinyl acetate content that is higher than that of the first EVA. (In other examples, the second region comprises a soft polymer that is not an EVA copolymer.)

The use of EVA with higher vinyl acetate content is advantageous, for example, in that it is more readily processable using various techniques (e.g., co-extrusion, over-lay extrusion, solvent-based coating and casting, solvent-based imbibing, etc.) than lower vinyl acetate content EVA. EVA with higher vinyl acetate content is also softer and thus potentially more comfortable than EVA with lower vinyl acetate content, particularly where disposed on an outer surface of the device.

In some embodiments, the first EVA is a low vinyl acetate content EVA while the second EVA is a high vinyl acetate content EVA.

In some embodiments, the vinyl acetate content of the second EVA is at least 5 wt % higher, at least 10 wt % higher, at least 15 wt % higher, at least 20 wt % higher, at least 25 wt % higher or even at least 30 wt % higher than the vinyl acetate content of the first EVA.

In some embodiments, the second region comprises a therapeutic agent. In certain of these embodiments, the first region either does not comprise a substantial amount of the therapeutic agent, or it comprises a lower concentration of the therapeutic agent than that of the second region. For example, in some instances, the average concentration of the therapeutic agent in the second region may be at least 2 times (e.g., ranging from 2 to 5 to 10 or more times) the average concentration found in the first region. In some instances, the therapeutic agent is present, if at all, only at the surface of the first region and does not extend substantially into the bulk of the first region.

In some embodiments, the second region comprises a therapeutic agent and a blend of two or more high vinyl acetate content EVA copolymers, each of different vinyl acetate content, thereby allowing further control over the release profile of the therapeutic agent.

In some embodiments, the second region is disposed around the first region, for example, in the form of a layer (e.g., a coated layer or a co-extruded layer) that wraps around the first region. In certain of these embodiments, the first region is in the form of an inner annular layer and the second region is in the form of an outer annular layer. One particular example of such an embodiment is described below in conjunction with FIGS. 3A and 3B.

The present inventors have found that high vinyl acetate content EVA has certain advantages relative to low vinyl acetate content EVA when it comes to therapeutic agent loading. Low vinyl acetate content EVA can be loaded with a therapeutic agent by mixing the therapeutic agent and the EVA when the EVA is in the melt stage (e.g., using an extruder or another device). However, this is an elevated temperature process which may adversely affect the stability of many therapeutic agents. Also, when the required loading quantity is relatively low, compounding loading is not preferred as the release rate could be limited by the limited amount of therapeutic agent available. Ambient (e.g., room) temperature methods are also available for creating therapeutic-agent-containing polymeric regions, including, for example, solvent-based methods in which (a) a polymeric region is formed from a solution containing at least one polymer, at least one therapeutic agent and at least one solvent species or (b) a previously formed polymeric region is exposed (e.g., by spraying, imbibing, etc.) to a solution containing at least one therapeutic agent and at least one solvent species (e.g., at least one solvent species that is capable of swelling, and/or dissolving the polymeric region). The present inventors have found that low vinyl acetate content EVA, unfortunately, does not lend itself to such solvent processing techniques, whereas high vinyl acetate content EVA does, because it generally more readily undergoes dissolution or swelling, for example, completely or partially dissolving and/or swelling in the presence of organic solvents. In various embodiments, a high vinyl acetate content EVA region is nonetheless used along with a low vinyl acetate content EVA, in order to enhance the physical properties of the device, for example, in order to increase tensile strength, elongation, pushability, handling and so forth. This combined approach also significantly increases the efficiency of loaded therapeutic agents through higher release rates.

Soft polymers other than high vinyl acetate content EVA that are readily imbibed can also be used as materials for the second region.

Soft polymers other than high vinyl acetate content EVA may be selected from synthetic and natural polymers, biodegradable and non-biodegradable polymers, hydrogel and non-hydrogel polymers. Specific examples include thermoplastic elastomers, for example, styrenic block copolymers such as poly(styrene-b-ethylene/butylene-b-styrene (SEBS) based materials, poly(styrene-b-butadiene-b-styrene) based materials, and poly(styrene-b-isobutylene-b-styrene (SIBS) based materials, thermoplastic polyurethanes including poly(ether-urethanes) such as Pellethane™, poly(aliphatic/aromatic-ester) (PED) multiblock copolymers composed of dimer fatty acid (DFA), poly(butylene terephthalate) (PBT), thermoplastic polyester elastomers, silicone based polymers, N-vinylpyrrolidone based polymers, acrylic based polymers, and chemically or physically crosslinked polysaccharide based materials (e.g., cellulose), protein based materials, peptide based materials, and other crosslinked hydrophilic polymers.

Examples of medical devices benefiting from the present invention include implantable or insertable medical devices, for example, catheters (e.g., urological or vascular catheters such as balloon catheters and various central venous catheters), guide wires, balloons, filters (e.g., vena cava filters and mesh filters for distil protection devices), stents (including coronary vascular stents, peripheral vascular stents, cerebral, urethral, ureteral, biliary, tracheal, gastrointestinal and esophageal stents), stent coverings, stent grafts, vascular grafts, abdominal aortic aneurysm (AAA) devices (e.g., AAA stents, AAA grafts), vascular access ports, dialysis ports, embolization devices including cerebral aneurysm filler coils (including Guglielmi detachable coils and metal coils), embolic agents, hermetic sealants, septal defect closure devices, myocardial plugs, patches, pacemakers, lead coatings including coatings for pacemaker leads, defibrillation leads, and coils, ventricular assist devices including left ventricular assist hearts and pumps, total artificial hearts, shunts, valves including heart valves and vascular valves, anastomosis clips and rings, cochlear implants, tissue bulking devices, and tissue engineering scaffolds for cartilage, bone, skin and other in vivo tissue regeneration, sutures, suture anchors, tissue staples and ligating clips at surgical sites, cannulae, metal wire ligatures, urethral slings, hernia “meshes”, artificial ligaments, orthopedic prosthesis such as bone grafts, bone plates, joint prostheses, orthopedic fixation devices such as interference screws in the ankle, knee, and hand areas, tacks for ligament attachment and meniscal repair, rods and pins for fracture fixation, screws and plates for craniomaxillofacial repair, dental implants, or other device that is implanted or inserted into the body.

The medical devices of the present invention thus include, for example, implantable and insertable medical devices that are used for systemic treatment, as well as those that are used for the localized treatment of any mammalian tissue or organ. Non-limiting examples are tumors; organs including the heart, coronary and peripheral vascular system (referred to overall as “the vasculature”), the urogenital system, including kidneys, bladder, urethra, ureters, prostate, vagina, uterus and ovaries, eyes, ears, spine, nervous system, lungs, trachea, esophagus, intestines, stomach, brain, liver and pancreas, skeletal muscle, smooth muscle, breast, dermal tissue, cartilage, tooth and bone.

In certain embodiments, the implantable or insertable medical devices of the invention are urological medical devices. Urological medical devices for use in conjunction with the present invention include any device which is suitable for at least partial insertion into the urinary tract of a subject, including the kidneys (e.g., in the renal calyx, renal pelvis, etc.), ureters, bladder and urethra. These include various elongated devices including elongated devices having any of a variety of solid and hollow cross-sections including circular (e.g., tubular and rod-shaped devices), oval, triangular, and rectangular (e.g., ribbon-shaped devices), among many other regular and irregular cross sections. Specific examples include urological stents, for example, urethral and ureteral stents, urological catheters (e.g., drainage catheters, guide catheters, etc.), guidewires, urological scopes (e.g., cytoscopes, ureteroscopes, nephroscopes, etc.), tissue engineering scaffolds, grafts, patches and synthetic meshes, among others.

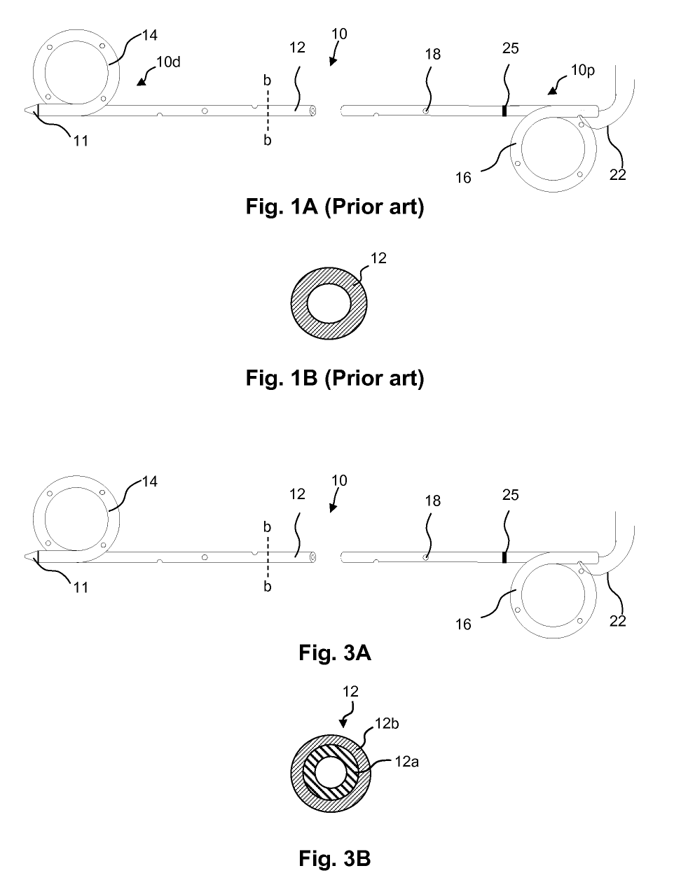

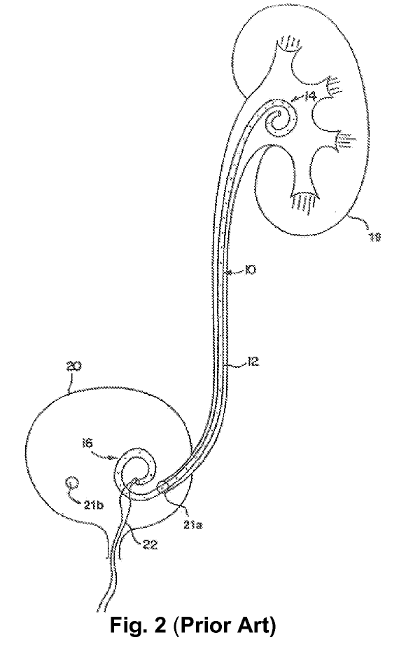

A ureteral stent in accordance with an embodiment of the present invention is schematically illustrated in FIGS. 3A and 3B. Like the stent of FIGS. 1A and 1B, the stent 10 is tubular, having a shaft 12, a distal (renal) retention structure (e.g., renal “pigtail” 14) and a proximal (bladder) retention structure (e.g., bladder “pigtail” 16). Other examples of retention structures for use in ureteral stents include, for example, spirals, coils, corkscrews, mallincotts, barbs, mushrooms and hook ends, among others. As with the stent of FIGS. 1A-B, the stent 10 of FIGS. 3A-B may be further provided with a tapered tip 11, multiple side ports 18 (one numbered), graduation marks 25 (one illustrated), and a suture 22. Unlike the stent of FIGS. 1A-B, however, the shaft 12 of stent 10 is a two-layer construction consisting of an inner annular layer 12a and an outer annular layer 12b, which can be seen from the schematic cross-sectional view of FIG. 3B (taken along line b-b of FIG. 3A).

In the particular embodiment shown, the inner annular layer 12a comprises a low vinyl acetate content EVA, while the outer annular layer 12b comprises a high vinyl acetate content EVA. Moreover, the outer annular layer 12b comprises a therapeutic agent, for example, an antimicrobial agent and/or an agent that reduces pain and discomfort, among other possibility.

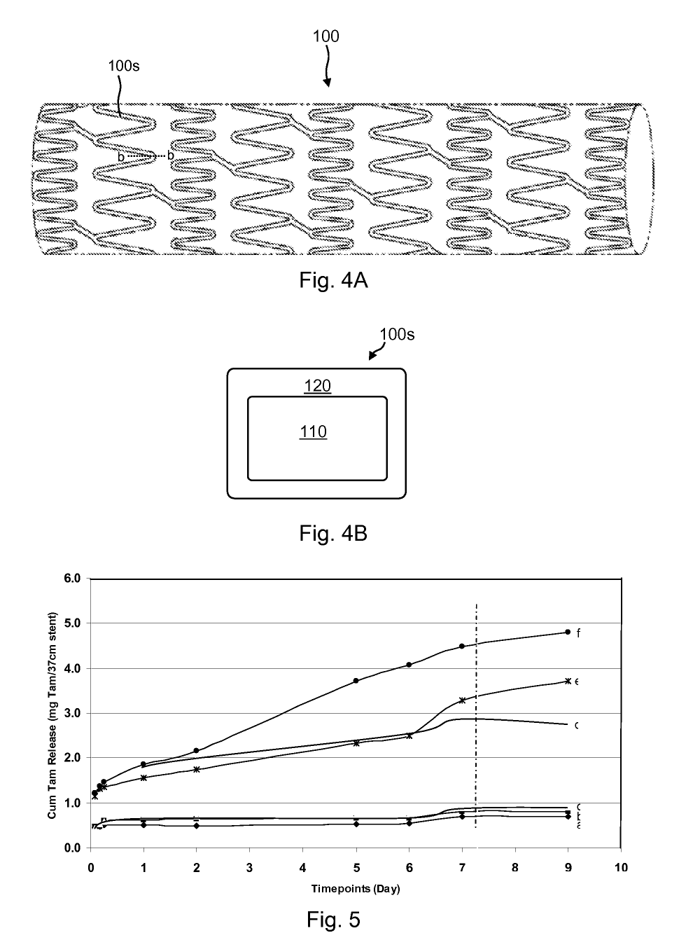

Although urological devices are described above, the invention is not so-limited. For example, other aspects of the invention pertain to vascular medical devices. A vascular stent in accordance with an embodiment of the present invention is schematically illustrated in FIGS. 4A and 4B. FIG. 4A shows a stent 100 which contains a number of interconnected struts 100s. FIG. 4B is a cross-section taken along line b-b of strut 110s of stent 100 of FIG. 4A, and shows a stainless steel stent substrate 110 and a coating 120, which encapsulates the substrate 110. The coating 120 is a low durometer layer that contains EVA (e.g., a low vinyl acetate content EVA) and a therapeutic agent (e.g., an antiproliferative agent for combating restenosis), which has been imbibed into the coating 120. Typical thicknesses of the coating 120 range from 1 to 50 μm.

Although devices with hollow lumens, specifically stents, are described in detail herein, the medical devices to which the principles of the invention may be applied include a wide range of implantable or insertable medical devices, as previously indicated.

As noted above, therapeutic agents for use in the medical devices of the invention include antimicrobial agents and agents that reduce pain and discomfort, such as antispasmodic agents, alpha-adrenergic blockers, corticosteroids, narcotic analgesic agents, non-narcotic analgesic agents, local anesthetic agents, and combinations thereof.

The term “antimicrobial agent” as used herein means a substance that kills and/or inhibits the proliferation and/or growth of microbes, particularly bacteria, fungi and yeast. Antimicrobial agents, therefore, include biocidal agents and biostatic agents as well as agents that possess both biocidal and biostatic properties. In the context of the present invention, the antimicrobial agent kills and/or inhibits the proliferation and/or growth of microbes on and around the surfaces of an implanted medical device, and can therefore inhibit biofilm formation (encrustation) in some cases.

Antimicrobial agents may be selected, for example, from triclosan, chlorhexidine, nitrofurazone, benzalkonium chlorides, silver salts and antibiotics, such as rifampin, gentamicin and minocycline, and combinations thereof, among others.

Further antimicrobial agents may be selected, for example, from suitable members of the following: the penicillins (e.g., penicillin G, methicillin, oxacillin, ampicillin, amoxicillin, ticarcillin, etc.), the cephalosporins (e.g., cephalothin, cefazolin, cefoxitin, cefotaxime, cefaclor, cefoperazone, cefixime, ceftriaxone, cefuroxime, etc.), the carbapenems (e.g., imipenem, metropenem, etc.), the monobactems (e.g., aztreonem, etc.), the carbacephems (e.g., loracarbef, etc.), the glycopeptides (e.g., vancomycin, teichoplanin, etc.), bacitracin, polymyxins, colistins, fluoroquinolones (e.g., norfloxacin, lomefloxacin, fleroxacin, ciprofloxacin, enoxacin, trovafloxacin, gatifloxacin, etc.), sulfonamides (e.g., sulfamethoxazole, sulfanilamide, etc.), diaminopyrimidines (e.g., trimethoprim, etc.), rifampin, aminoglycosides (e.g., streptomycin, neomycin, netilmicin, tobramycin, gentamicin, amikacin, etc.), tetracyclines (e.g., tetracycline, doxycycline, demeclocycline, minocycline, etc.), spectinomycin, macrolides (e.g., erythromycin, azithromycin, clarithromycin, dirithromycin, troleandomycin, etc.), and oxazolidinones (e.g., linezolid, etc.), among others, as well as combinations and pharmaceutically acceptable salts, esters and other derivatives of the same.

Antispasmodic agents may be selected, for example, from suitable members of the following: alibendol, ambucetamide, aminopromazine, apoatropine, bevonium methyl sulfate, bietamiverine, butaverine, butropium bromide, n-butylscopolammonium bromide, caroverine, cimetropium bromide, cinnamedrine, clebopride, coniine hydrobromide, coniine hydrochloride, cyclonium iodide, difemerine, diisopromine, dioxaphetyl butyrate, diponium bromide, drofenine, emepronium bromide, ethaverine, feclemine, fenalamide, fenoverine, fenpiprane, fenpiverinium bromide, fentonium bromide, flavoxate, flopropione, gluconic acid, guaiactamine, hydramitrazine, hymecromone, leiopyrrole, mebeverine, moxaverine, nafiverine, octamylamine, octaverine, oxybutynin chloride, pentapiperide, phenamacide hydrochloride, phloroglucinol, pinaverium bromide, piperilate, pipoxolan hydrochloride, pramiverin, prifinium bromide, properidine, propivane, propyromazine, prozapine, racefemine, rociverine, spasmolytol, stilonium iodide, sultroponium, tiemonium iodide, tiquizium bromide, tiropramide, trepibutone, tricromyl, trifolium, trimebutine, n,n-1trimethyl-3,3-diphenyl-propylamine, tropenzile, trospium chloride, and xenytropium bromide, among others, as well as combinations and pharmaceutically acceptable salts, esters and other derivatives of the same.

Examples of alpha-adrenergic blockers for use in the present invention may be selected from suitable members of the following: alfuzosin, amosulalol, arotinilol, dapiprazole, doxazosin, ergoloid mesylates, fenspiride, idazoxan, indoramin, labetalol, manotepil, naftopidil, nicergoline, prazosin, tamsulosin, terazosin, tolazoline, trimazosin, and yohimbine, among others, as well as combinations and pharmaceutically acceptable salts, esters and other derivatives of the same. Of these, tamsulosin, alfuzosin, doxazosin, prazosin, tamsulosin and terazosin are alpha-1-adrenergic blockers, of which tamsulosin and alfuzosin are selective alpha-1-adrenergic blockers.

Examples of corticosteroids for use in the present invention may be selected from suitable members of the following: betamethasone, cortisone, dexamethasone, deflazacort, hydrocortisone, methylprednisolone, prednisolone, prednisone and triamcinolone, among others, as well as combinations and pharmaceutically acceptable salts, esters and other derivatives of the same.

Examples of narcotic analgesic agents for use in the present invention may be selected from suitable members of the following: codeine, morphine, fentanyl, meperidine, propoxyphene, levorphanol, oxycodone, oxymorphone, hydromorphone, pentazocine, and methadone, among others, as well as combinations and pharmaceutically acceptable salts, esters and other derivatives of the same.

Examples of non-narcotic analgesic agents for use in the present invention may be selected from suitable members of the following: analgesic agents such as acetaminophen, and non-steroidal anti-inflammatory drugs such as aspirin, diflunisal, salsalate, ibuprofen, ketoprofen, naproxen indomethacin, celecoxib, valdecoxib, diclofenac, etodolac, fenoprofen, flurbiprofen, ketorolac, meclofenamate, meloxicam, nabumetone, naproxen, oxaprozin, piroxicam, sulindac, tolmetin, and valdecoxib, among others, as well as combinations and pharmaceutically acceptable salts, esters and other derivatives of the same.

Examples of local anesthetic agents for use in the present invention may be selected from suitable members of the following: benzocaine, cocaine, lidocaine, mepivacaine, and novacaine, among others, as well as combinations and pharmaceutically acceptable salts, esters and other derivatives of the same.

Further examples of therapeutic agents for use in the present invention may be selected from suitable members of the following: (a) anti-thrombotic agents such as heparin, heparin derivatives, urokinase, clopidogrel, and PPack (dextrophenylalanine proline arginine chloromethylketone); (b) anti-inflammatory agents such as dexamethasone, prednisolone, corticosterone, budesonide, estrogen, sulfasalazine and mesalamine; (c) antineoplastic/antiproliferative/anti-miotic agents such as paclitaxel, 5-fluorouracil, cisplatin, vinblastine, vincristine, epothilones, endostatin, angiostatin, angiopeptin, monoclonal antibodies capable of blocking smooth muscle cell proliferation, and thymidine kinase inhibitors; (d) anesthetic agents such as lidocaine, bupivacaine and ropivacaine; (e) anti-coagulants such as D-Phe-Pro-Arg chloromethyl ketone, an RGD peptide-containing compound, heparin, hirudin, antithrombin compounds, platelet receptor antagonists, anti-thrombin antibodies, anti-platelet receptor antibodies, aspirin, prostaglandin inhibitors, platelet inhibitors and tick antiplatelet peptides; (f) vascular cell growth promoters such as growth factors, transcriptional activators, and translational promotors; (g) vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; (h) protein kinase and tyrosine kinase inhibitors (e.g., tyrphostins, genistein, quinoxalines); (i) prostacyclin analogs; (j) cholesterol-lowering agents; (k) angiopoietins; (l) antimicrobial agents such as triclosan, cephalosporins, aminoglycosides and nitrofurantoin; (m) cytotoxic agents, cytostatic agents and cell proliferation affectors; (n) vasodilating agents; (o) agents that interfere with endogenous vasoactive mechanisms; (p) inhibitors of leukocyte recruitment, such as monoclonal antibodies; (q) cytokines; (r) hormones; (s) inhibitors of HSP 90 protein (i.e., Heat Shock Protein, which is a molecular chaperone or housekeeping protein and is needed for the stability and function of other client proteins/signal transduction proteins responsible for growth and survival of cells) including geldanamycin, (t) smooth muscle relaxants such as alpha receptor antagonists (e.g., doxazosin, tamsulosin, terazosin, prazosin and alfuzosin), calcium channel blockers (e.g., verapimil, diltiazem, nifedipine, nicardipine, nimodipine and bepridil), beta receptor agonists (e.g., dobutamine and salmeterol), beta receptor antagonists (e.g., atenolol, metaprolol and butoxamine), angiotensin-II receptor antagonists (e.g., losartan, valsartan, irbesartan, candesartan, eprosartan and telmisartan), and antispasmodic/anticholinergic drugs (e.g., oxybutynin chloride, flavoxate, tolterodine, hyoscyamine sulfate, diclomine), (u) bARKct inhibitors, (v) phospholamban inhibitors, (w) Serca 2 gene/protein, (x) immune response modifiers including aminoquizolines, for instance, imidazoquinolines such as resiquimod and imiquimod, (y) human apolioproteins (e.g., AI, AII, AIII, AIV, AV, etc.), (z) selective estrogen receptor modulators (SERMs) such as raloxifene, lasofoxifene, arzoxifene, miproxifene, ospemifene, PKS 3741, MF 101 and SR 16234, (aa) PPAR agonists such as rosiglitazone, pioglitazone, netoglitazone, fenofibrate, bexaotene, metaglidasen, rivoglitazone and tesaglitazar, (bb) prostaglandin E agonists such as alprostadil or ONO 8815Ly, (cc) thrombin receptor activating peptide (TRAP), (dd) vasopeptidase inhibitors including benazepril, fosinopril, lisinopril, quinapril, ramipril, imidapril, delapril, moexipril and spirapril, and (ee) thymosin beta 4, among others, as well as combinations and pharmaceutically acceptable salts, esters and other derivatives of the same.

Still further examples of therapeutic agents for use in the present invention may be selected from suitable members of the following: ketorolac and pharmaceutically acceptable salts thereof (e.g., the tromethamine salt thereof), oxybutynin chloride, triclosan, taxanes such as paclitaxel (including particulate forms thereof, for instance, protein-bound paclitaxel particles such as albumin-bound paclitaxel nanoparticles, e.g., ABRAXANE), olimus family compounds such as sirolimus, everolimus, tacrolimus, and zotarolimus, Epo D, dexamethasone, estradiol, halofuginone, cilostazole, geldanamycin, ABT-578 (Abbott Laboratories), trapidil, liprostin, Actinomcin D, Resten-NG, Ap-17, abciximab, clopidogrel, Ridogrel, beta-blockers, bARKct inhibitors, phospholamban inhibitors, Serca 2 gene/protein, imiquimod, human apolioproteins (e.g., AI-AV), and growth factors (e.g., VEGF-2), as well as combinations and pharmaceutically acceptable salts, esters and other derivatives of the same.

Many of the above and other therapeutic agents may be found, for example, in The Merck Index, 13^{th} Edition, M. J. O'Neil, Senior Editor, published by Merck Research Laboratories, 2001.

A wide range of agent loadings may be used in conjunction with the medical devices of the present invention. The amount of therapeutic agent present, will depend, for example, upon the efficacy of the therapeutic agent employed, the release rate, and so forth. One skilled in the art can readily determine an appropriate therapeutic agent loading to achieve the desired outcome. Typical loadings within the polymeric regions of the invention can range, for example, from than 1 wt % or less to 2 wt % to 5 wt % to 10 wt % to 25 wt % to 50 wt % or more.

In certain embodiments, the device may exhibit an extended release profile. By “extended release profile” is meant a release profile by which a pharmaceutically effective amount of therapeutic agent continues to be released at least one day 1 after device implantation or insertion, for example, from 1 day to 2 days to 4 days to 1 week to 2 weeks to 1 month to 2 months to 6 months to 1 year or more after device implantation.

The medical device of the present invention may also contain one or more further optional additives, for example, selected from radio-opacifying agents, pigments, or other additives such as plasticizers, extrusion lubricants, polymers other than or in addition to EVA, and combinations of the above, among others, in amounts effective to serve their intended purposes.

Radio-opacifying agents facilitate viewing of the medical device during insertion of the device and at any point while the device is implanted. A radio-opacifying agent typically functions by scattering x-rays. The areas of the medical device that scatter the x-rays are detectable on a radiograph. Among radio-opacifying agents useful in the medical device of the present invention are included bismuth salts such as bismuth subcarbonate, bismuth oxychloride, bismuth trioxide, barium sulfate, tungsten, and mixtures thereof, with bismuth salts typically being preferred. Where present, the radio-opacifying agent is typically present in an amount of from about 10% to about 40% (including 10% to 15% to 20% to 25% to 30% to 35% to 40%, with 15-30% being more typical). One skilled in the art can readily determine an appropriate radio-opacifying agent content to achieve the desired visibility.

In some embodiments, the devices of the invention are optionally provided with lubricious layers.

Devices in accordance with the present invention can be made using various manufacturing techniques.

In some embodiments of the invention, regions (e.g., layers, monolithic structures, etc.) containing a soft polymer, for example, high vinyl acetate content EVA, among others, and a therapeutic agent are created using solvent-based techniques. For example, a region can be formed by (a) first providing a solution or dispersion that contains (i) a solvent system, (ii) at least one soft polymer (e.g., one or more high vinyl acetate content EVA copolymers), (iii) at least one therapeutic agent and (iv) any further optional agent(s), and (b) subsequently removing the solvent. The solvent system that is ultimately selected will contain one or more solvent species (e.g., one or more organic solvents such as toluene, tetrahydrofuran, chloroform), which are generally selected based on their ability to dissolve the polymer(s) that form the polymeric region, as well as the therapeutic agent(s) and any optional supplemental agent(s) that may be present. In addition to other factors may be considered, including drying rate, surface tension, etc. Preferred solvent-based techniques include solvent casting techniques, dipping techniques, solvent spraying techniques, spin coating techniques, web coating techniques, coating techniques involving coating via mechanical suspension including air suspension, ink jet techniques, electrostatic coating techniques, and combinations of these processes, among others.

In other embodiments, a polymer-containing region, for example, an EVA-containing region (e.g., a high and/or low vinyl acetate content EVA), among others, is created without a therapeutic agent (e.g., using a thermoplastic-based method, a solvent-based method, etc.). In some of these embodiments (e.g., in the case of a high vinyl acetate content EVA), formation of the polymer-containing region is followed by incorporation of a therapeutic agent (e.g., using a solvent-based method). For example, a polymer-containing region may be formed by (a) first providing a solution or dispersion that contains (i) a solvent system, (ii) one or more polymers (e.g., one or more high vinyl acetate content EVA copolymers), and (iii) any optional supplemental agent(s), and (b) subsequently removing the solvent. As another example, a polymer-containing region may be formed by (a) first providing a polymer melt that contains (i) one or more polymers (e.g., one or more low or high vinyl acetate content EVA copolymers), and (ii) any optional supplemental agent(s), and (b) subsequently cooling the melt.

In certain embodiments, a polymer-containing solution (where solvent-based processing is employed) or a polymer melt (where thermoplastic processing is employed) is applied to a substrate to form a polymeric region. For example, the substrate can correspond to all or a portion of an implantable or insertable medical device body to which a polymeric region is applied. For example, a high vinyl acetate content EVA may be applied to a low vinyl acetate content EVA in this manner. The substrate can also be, for example, a template, such as a mold, from which the polymeric region is removed after the molding process (e.g., compression molding, blow molding, rotational molding and injection molding including co-injection or sequential injection molding technology such as laminar injection molding, LIM, technology, where multilayer structures are desired, etc.). In certain other embodiments, for example, spinning and extrusion techniques (e.g., extrusion into extrusion into sheets, fibers, rods and tubes, including co-extrusion, multi-layer extrusion, multi-lumen extrusion, etc.), one or more polymeric regions can be formed without the aid of a substrate. For example, a tubular extrusion with concentric high and low vinyl acetate content EVA regions may be formed in this manner.

Thus, using the above and other processing techniques, entire devices or portions thereof can be formed. In one specific example, an entire stent body may be extruded. In another specific example, a multilayer stent body may be co-extruded. In another, a polymeric layer may be provided by forming a coating layer onto a pre-existing stent body. In yet another specific example, a stent body may be cast in a mold.

Where a therapeutic agent is to be introduced into a pre-existing polymer-containing region (e.g., one containing one or more high vinyl acetate content EVA copolymers or another soft polymer), the therapeutic agent may first be dissolved in a solvent system (made up of one or more organic solvent species) and the resulting therapeutic-agent-containing solution brought into contact with the polymer-containing region. In certain of these embodiments (e.g., those in which the contact time is relatively short such that equilibrium conditions are not established), this process will result in a concentration gradient in which the concentration is greatest at the surface and decreases as one proceeds into the bulk in a direction that is normal to the surface. The solvent systems in these embodiments are generally selected based on their ability to dissolve the therapeutic agent and to dissolve and/or swell the polymer-containing region, among other factors. The therapeutic-agent-containing solution may be brought into contact with the polymer-containing region using various techniques, including dipping techniques, solvent spraying techniques, spin coating techniques, web coating techniques, coating techniques involving coating via mechanical suspension including air suspension, ink jet techniques, electrostatic coating techniques, and combinations of these processes, among others.

In certain embodiments, a therapeutic agent is introduced to a pre-existing polymer-containing region in this fashion by the device manufacturer during the device manufacturing process. In certain other embodiments, prior to insertion or implantation, health care professional (e.g., a physician or a physician's assistant) may introduce a therapeutic agent in this fashion (e.g., by spraying the device with, or dipping the device into, a therapeutic-agent-containing solution) thereby loading the device with a therapeutic agent of choice. These embodiments are advantageous, for example, in that the medical devices are not dedicated to a single therapeutic agent only, as they can be imbibed with different kinds of drugs.

In certain embodiments of the invention, kits are provided which comprise a medical device with a polymer-containing region like those described herein, along with a solution comprising a therapeutic agent dissolved in a solvent that is also capable of dissolving and/or swelling the polymer-containing region (e.g., disposed within a container such as a vial, etc.).

In some embodiments, processing may comprise dry blending, compounding or otherwise mixing one or more polymers and one or more optional additives to form a relatively homogeneous mixture thereof and then forming a polymeric region from the homogenous mixture. Mixing or compounding polymer(s) with one or more optional additives to form a relatively homogeneous mixture thereof may be performed with any device known in the art and useful for mixing polymeric materials with additives. Where the polymer(s) are thermoplastic in nature, the additives may be mixed with the polymer(s) while in a melt stage to form a relatively homogenous mixture. A common way of doing so is to apply mechanical shear to a mixture of the polymer(s) and optional additives. Mixing may also be achieved by dissolving polymer(s) of interest with one or more optional additives in a suitable solvent system.

As a specific example, a ureteral stent may be formed, which contains (a) an inner layer comprising (i) a radio-opacifying agent, (ii) a colorant and (iii) the balance ethylene vinyl acetate (EVA) copolymer (e.g., Elvax® 460, from DuPont) and (b) an outer layer comprising ethylene vinyl acetate (EVA) copolymer (e.g., Elvax® 250, from DuPont). First, the Elvax® 460 may be compounded with the additional non-polymeric additives (radio-opacifying agent, colorant) and extruded into pellets. Then these pellets and pellets of Elvax® 250 may then be co-extruded into a multi-layer tubular structure having inner and outer layers like that shown in cross-section in FIG. 3B, where the Elvax® 460 containing material forms the inner layer and the Elvax® 250 containing material forms the outer layer. (Alternatively, a firm tube can be first extruded using Elvax® 460, followed by extrusion of the softer Elvax® 250 over the firm tube.) Being soft, the Elvax® 250 containing material can function as a soft cushion to increase comfort. Also, where transparent, the Elvax® 250 containing material can significantly enhance the color of the core material. The extruded tube may be subsequently be cut, provided with a tapered tip, annealed, machined to form side ports, heat-treated for pigtail formation, and a suture added. The Elvax® 250 containing layer may be preferentially loaded with a therapeutic agent (relative to the Elvax® 460 containing layer) by exposing (e.g., by dipping, spraying, etc.) the stent to a solution containing a therapeutic agent and one or more solvent species (e.g., one in which the drug is readily dissolved and which is able to swell the polymer and imbibe or adsorb the drug, for instance, a solution of tamsulosin in chloroform).

Once a medical device is formed, it is typically packaged and sterilized. Implantable or insertable medical devices are commonly sterilized by exposure to ethylene oxide or to radiation. For example, the medical device may be placed in a foil pouch, which is either evacuated or is provided with an inert atmosphere (e.g., an atmosphere of nitrogen and/or noble gases such as argon, etc.), and the pouch may subsequently be exposed to electron beam radiation as is known in the art.

Various aspects of the invention of the invention relating to the above are enumerated in the following paragraphs:

Aspect 1. A medical device configured for at least partial implantation or insertion into a subject, the medical device comprising a first region comprising a first poly(ethylene-co-vinyl acetate) (EVA) and a second region adjacent to the first region and disposed around the first region that comprises a second EVA that has a higher vinyl acetate content than the first EVA.

Aspect 2. The medical device of aspect 1, wherein the first region comprises a durometer ranging from 80 to 100 (Shore A).

Aspect 3. The medical device of aspect 1, wherein the first region comprises a vinyl acetate content ranging from 1 wt % to 35 wt %.

Aspect 4. The medical device of aspect 1, wherein the second region comprises a durometer ranging from 65 to 83 (Shore A).

Aspect 5. The medical device of aspect 1, wherein the second region comprises a vinyl acetate content ranging from 25 to 40 wt %.

Aspect 6. The medical device of aspect 1, wherein the second region comprises a plurality of EVA polymers each having a durometer ranging from 65 to 83.

Aspect 7. The medical device of aspect 1, wherein the medical device is a urological medical device.

Aspect 8. The medical device of aspect 7, wherein the second region comprises a therapeutic agent selected from antimicrobial agents and agents that reduce pain and discomfort.

Aspect 9. The medical device of aspect 7, wherein the second region comprises a therapeutic agent selected from antibiotics, antispasmodic agents, alpha-adrenergic blockers, corticosteroids, narcotic analgesic agents, non-narcotic analgesic agents and local anesthetic agents.

Aspect 10. The medical device of aspect 7, wherein the second region comprises tamsulosin.

Aspect 11. The medical device of aspect 1, wherein the medical device is an elongated medical device.

Aspect 12. The medical device of aspect 1, wherein the medical device is a tubular medical device and wherein the first and second regions are concentric first and second annular layers.

Aspect 13. The medical device of aspect 12, wherein the first and second annular layers are extruded layers.

Aspect 14. The medical device of aspect 12, wherein the second annular layer is solvent coated over the first layer.

Aspect 15. The medical device of aspect 12, wherein the first annular layer comprises said first EVA and a radio-opacifying agent, and wherein the second annular layer comprises said second EVA and a therapeutic agent.

Aspect 16. The medical device of aspect 12, wherein the medical device is a ureteral stent.

Aspect 17. A kit comprising (a) the medical device of aspect 1 and (b) a solution comprising a therapeutic agent and a solvent that swells the second region upon contacting the second region.

Aspect 18. The kit of aspect 17, wherein the solvent system comprises solvent species selected from toluene, tetrahydrofuran, chloroform and combinations thereof.

Aspect 19. A method of loading a medical device comprising contacting the medical device of aspect 1 with a solution comprising a therapeutic agent and a solvent that swells the second region upon contacting the second region.

Aspect 20. An implantable or insertable medical device comprising (a) a substrate and (b) a polymeric layer that comprises high vinyl acetate content EVA and an imbibed therapeutic agent.

Aspect 21. A medical device configured for at least partial implantation or insertion into a subject, the medical device comprising (a) a first region comprising a poly(ethylene-co-vinyl acetate) (EVA) and (b) a second region adjacent to the first region and disposed around the first region that comprises (i) a second polymer that is softer than the EVA and (ii) a therapeutic agent.

### EXAMPLE 1

Samples (pigtailed or straight in shape and sized between 22 to 28 cm by length and from 4 Fr to 14 Fr in outside diameter) of a soft EVA (Elvax® 250) and a hard EVA (Elvax® 460) were exposed to a solution containing 20 mg/ml tamsulosin in 30:70 methanol:chloroform between 1 to 30 minutes. Tamsulosin release was evaluated in 10 mmol phosphate buffered saline (PBS). Cumulative release as a function of time is shown for these samples in FIG. 5 (curve a, Elvax® 460 imbibed for 5 min; curve b, Elvax® 460 imbibed for 15 min; curve c, Elvax® 460 imbibed for 25 min; curve d, Elvax® 250 imbibed for 5 min; curve e, Elvax® 250 imbibed for 15 min; curve f, Elvax® 250 imbibed for 25 min). As can be seen from FIG. 5, it is possible to load EVA with drug using an imbibing technique, with the soft EVA being much more readily loaded than then hard EVA. Moreover, longer imbibing times resulted in increased drug loading, particularly for the soft EVA.

Although various embodiments are specifically illustrated and described herein, it will be appreciated that modifications and variations of the present invention are covered by the above teachings and are within the purview of any appended claims without departing from the spirit and intended scope of the invention.

1. A medical device configured for at least partial implantation or insertion into a subject, said medical device comprising a first region comprising a first poly(ethylene-co-vinyl acetate) (EVA) and a second region adjacent to the first region and disposed around the first region that comprises a second EVA that has a higher vinyl acetate content than the first EVA.

2. The medical device of claim 1, wherein said first region comprises a durometer ranging from 80 to 100 (Shore A).

3. The medical device of claim 1, wherein said first region comprises a vinyl acetate content ranging from 1 wt % to 35 wt %.

4. The medical device of claim 1, wherein said second region comprises a durometer ranging from 65 to 83 (Shore A).

5. The medical device of claim 1, wherein said second region comprises a vinyl acetate content ranging from 25 to 40 wt %.

6. The medical device of claim 1, wherein said second region comprises a plurality of EVA polymers each having a durometer ranging from 65 to 83.

7. The medical device of claim 1, wherein said medical device is a urological medical device.

8. The medical device of claim 7, wherein said second region comprises a therapeutic agent selected from antimicrobial agents and agents that reduce pain and discomfort.

9. The medical device of claim 7, wherein said second region comprises a therapeutic agent selected from antibiotics, antispasmodic agents, alpha-adrenergic blockers, corticosteroids, narcotic analgesic agents, non-narcotic analgesic agents and local anesthetic agents.

10. The medical device of claim 7, wherein said second region comprises tamsulosin.

11. The medical device of claim 1, wherein said medical device is an elongated medical device.

12. The medical device of claim 1, wherein said medical device is a tubular medical device and wherein said first and second regions are concentric first and second annular layers.

13. The medical device of claim 12, wherein said first and second annular layers are extruded layers.

14. The medical device of claim 12, wherein said second annular layer is solvent coated over the first layer.

15. The medical device of claim 12, wherein said first annular layer comprises said first EVA and a radio-opacifying agent, and wherein said second annular layer comprises said second EVA and a therapeutic agent.

16. The medical device of claim 12, wherein said medical device is a ureteral stent.

17. A kit comprising
(a) said medical device of claim 1, and
(b) a solution comprising a therapeutic agent and a solvent that swells said second region upon contacting said second region.

18. The kit of claim 17, wherein said solvent system comprises solvent species selected from toluene, tetrahydrofuran, chloroform and combinations thereof.

19. A method of loading a medical device comprising contacting said medical device of claim 1 with a solution comprising a therapeutic agent and a solvent that swells said second region upon contacting said second region.

20. An implantable or insertable medical device comprising
(a) a substrate, and
(b) a polymeric layer that comprises high vinyl acetate content EVA and an imbibed therapeutic agent.

21. A medical device configured for at least partial implantation or insertion into a subject, said medical device comprising
(a) a first region comprising a poly(ethylene-co-vinyl acetate) (EVA), and
(b) a second region adjacent to said first region and disposed around said first region that comprises (i) a second polymer that is softer than said EVA and (ii) a therapeutic agent.
